(19) United States
(12) Patent Application Publication
(10) Pub. No.: US 2022/0054402 A1
(43) Pub. Date: Feb. 24, 2022

(54) COMPOSITIONS AND METHODS FOR EXTRACTING, STABILIZING, AND MANUFACTURING STABLE DOSAGE FORMS OF PSILOCIN, PSYCHEDELIC DRUGS, ENTHEOGENS, AND MEDICINAL MUSHROOMS AS NANO-DIMENSIONAL DRUG DELIVERY STRUCTURES

(71) Applicant: RICHARD C KAUFMAN, Santa Monica, CA (US)

(72) Inventor: RICHARD C KAUFMAN, Santa Monica, CA (US)

(21) Appl. No.: 17/518,091

(22) Filed: Nov. 3, 2021

Related U.S. Application Data
(60) Provisional application No. 63/109,912, filed on Nov. 5, 2020.

(51) Int. Cl. A61K 9/00; A61K 36/07

(52) U.S. Cl. CPC A61K 9/006; A61K 36/07; B82Y 5/00; A61K 2236/33; A61K 2236/50

(57) ABSTRACT

This invention discloses nano-dimensional compositions of psychedelic drugs, entheogens, and medicinal mushrooms formed from lipids, polymers, stabilizers, mucoadhesives, permeation enhancers, and chemical stabilizers for delivering stable dosage forms by intraoral, gastrointestinal, transdermal, and intranasal, routes of administration. Disclosed are unified product extraction and manufacturing of stable nano-dimensional dosage forms of psychedelic drugs, entheogen, and medicinal mushrooms, and methods for improving their pharmacokinetics, pharmacodynamics, and efficacy. Also disclosed are methods for dephosphorylating the precursor psilocybin into active psilocin molecules, and fast-acting, nano-dimensional structures of psychedelic drugs taken by intranasal and intraoral routes of administration producing activity onset in less than 10 minutes.

### CROSS-REFERENCE TO RELATED APPLICATIONS

This application claims priority from the U.S. provisional patent application Ser. No. 63/109,912, filed on Nov. 5, 2020, which is incorporated herein by reference in its entirety.

### TECHNICAL FIELD

The present invention relates to natural product extraction processes for isolating, separating, and stabilizing psychedelic drugs, or entheogen or medicinal mushroom, formulating them into nano-dimensional drug delivery structures, and improving their pharmacokinetics.

Aspects of the invention relate to compositions and methods for delivering nano-dimensional structures of psychedelic drugs, or entheogens, or medicinal mushrooms by intraoral, or intranasal, or transdermal, or gastrointestinal routes of administration.

Further aspects of the invention relate to compositions and methods for dephosphorylating psilocybin into psilocin and delivering fast-acting nano-dimensional-structures by intranasal or intraoral routes of administration.

### BACKGROUND

### Psychedelic Mushrooms

Psilocybin (PY, 4-phosphoryloxy-N,N-dimethyltryptamine) is the main psychoactive ingredient of hallucinogenic mushrooms and the precursor to biological active psilocin. Psilocin is present in the Psilcybe cubensis mushrooms in smaller amounts.

Psilocybin is a prodrug of psilocin. In vivo psilocybin molecules are metabolized into psilocin by dephosphorylation. After ingesting psilocybin, stomach acid and alkaline phosphatases in the small intestine cleave the phosphoryl ester bond from psilocybin. This changes water-soluble psilocybin into lipophilic psilocin which can cross the blood-brain barrier and produce its psychoactive effects.

Psilocin is a substituted tryptamine alkaloid that causes psychedelic effects. It is a chemical analog of the neurotransmitter serotonin. Receptor binding studies have found that psilocin is the primary bioactive psychoactive component based on its binding affinity at the serotonin 5-HT2A receptor. Psilocin has more than 200 times greater affinity than psilocybin for binding the serotonin 5-HT2A receptor.

Psilocin is highly unstable and degrades quickly in the presence of air, heat, and/or light. Psilocin is relatively unstable in alkaline solutions due to its phenolic hydroxy (—OH) group.

### Psychedelic Drugs

Psilocin and Psilocybin belong to a class of emerging “psychedelic drugs” under development in labs around the world. Psychedelic medicine drug classes include tryptamines, phenethylamines, and lysergamides. Some of the psychedelic drugs being researched for therapy include psilocybin, psilocin LSD (Lysergic acid diethylamide), DMT (Diemethyltryptamine), Ibogaine, mescaline, and MDMA.

Many psychedelic drugs and entheogens that are derived from and extracted from mushrooms, fungi, plants, botanicals, animals, flora, or synthesized in the laboratory are highly unstable. They can degrade over time after manufacturing and during storage before their use. Degradation appears more problematic for drugs made from natural product drug extraction techniques. Many psychedelic drugs, entheogens, and medicinal mushroom extracts have low intestinal bioavailability and have difficulty passing across the blood-brain barrier.

### Medicinal Mushrooms

Medicinal mushrooms are macroscopic fungi, used for disease prevention, therapeutic application, and nutritional reasons. There are more than 50 species of mushrooms with therapeutic properties, which are referred to as medicinal mushrooms.

Each species of medicinal mushroom contains a variety of biologically active phytochemicals and compounds. Among the most important are polysaccharides, such as β-glucans, which are present in all mushroom species. There are also species-specific terpenoids, triterpenes, polyphenols, flavonoids, carotenoids, proteins, fats, minerals, glycosides, alkaloids, volatile oils, tocopherols, vitamins, organic acids, and other phytochemicals.

Medicinal mushrooms generally strengthen the immune system (from their glucans) and exhibit anticancer properties. Species have demonstrated antioxidant, anti-inflammatory, and anti-allergenic properties. Some species can lower blood sugar, cholesterol levels, and blood pressure. Others help prevent diabetes, hypertension, cardiovascular disease, viral infections, and bacterial infections.

The most important medical mushroom species are Hericium erinaceus, Ganoderma lucidum, Trametes versicolor, Grifola frondosa, Lentinula edodes, Agaricus blazei, Cordyceps sinensis, Fomitopsis pinicola, Coprinus comatus, Monascus purpureus, Polyporus umbellatus, Piptoporus betulinus, and Pleurotus ostreatus.

The commercially available dosage forms of medicinal mushrooms have problems that disqualify them as effective for therapeutic use.

Whole Mushroom Powders have extremely low oral bioavailability. Their mycelium and fruiting bodies contain indigestible fibrous “chitin,” which surrounds and embeds the therapeutically active phytochemicals rendering mushroom powders difficult to absorb from the intestinal tract.

Mushroom Biomass Powder's bioactive phytochemicals have very low bioavailability due to their high amounts of indigestible chitin rendering them mostly ineffective.

Medicinal Mushroom Powder Extract's bioactive phytochemicals are often unstable and degrade in commercial dosage forms. They routinely have low solubility and slow rates of absorption.

Liquid Mushroom Extracts can have the same low bioavailability as powder extracts since 80 to 95% of their volume is solvent, alcohol, and water. These leaves only 5-20% of their volume as a mushroom extract. This leads to overpriced and ineffective formulations for therapeutic applications.

Ethanol-based Tinctures have a series of problems making them ineffective for therapeutic use. Their therapeutic β-glucans, proteins, and lipophilic phytochemicals are insoluble in ethanol forming the majority of tinctures volume majority soluble. are not normally soluble in tinctures. 80 to 95% of a tincture product volume is liquid solvent.

These problems prevent delivering doses of Medicinal Mushrooms in commercial products that are effective in therapy.

### Entheogens

Entheogens are psychoactive, hallucinogenic, psychedelic compounds or preparations derived from plants, fungi, or animals that have historically been used in religious, spiritual, or ritualistic practices. Each entheogen contains different biologically active phytochemicals and compounds that produce its experiential, psychoactive, and psychedelic effects.

Some of the better-known entheogens are cactus or extracted materials tested for natural phenethylamine compounds such as Peyote, San Pedro Cactus, Peruvian Torch, and Bolivian Torch. Mushroom or extracted materials tested for natural tryptamine compounds such as Psilocybe fungi. Ibogaine or extracted materials tested for natural tryptamine compounds such as Tabernanthe iboga and Trachelospermum jasminoides. DMT or extracted materials tested for natural tryptamine compounds such as Psychotria viridis, and Ayahuasca plant preparations. Other examples of entheogens include Acacia species, Kava lactones, ibotenic acid, Muscimol, Amanita muscarita, and Ololiuqui.

Similar to medicinal mushrooms, entheogens often have low oral bioavailability. They require a high milligram and gram dosage amount to be effective. There are sparse methods for modern preparation, standard compositions of active phytochemicals, and acceptable drug delivery dosage forms of entheogens at this time. Plus, like mushrooms, their active phytochemicals can degrade especially when extracted.

### SUMMARY OF THE EMBODIMENTS

This disclosure teaches compositions and methods of natural product extraction, isolation, separation, dephosphorylation, stabilizing and manufacturing dosage forms of psilocin, or psilocybin, or psychedelic drugs, or entheogens, or medicinal mushrooms; formulating a nano-dimensional-drug delivery system structure of lipids, polymers, stabilizers, mucoadhesives, permeation enhancers, chemical stabilizers, surfactants, and a psychedelic drug, or entheogen, or medicinal mushroom; natural product extraction processes for the isolation, separation, and stabilization of a psychedelic drug, or entheogen, or medicinal mushroom and their incorporation into a nano-dimensional drug delivery structure; nano-dimensional drug delivery system structures for improving the pharmacokinetics characteristics of a psychedelic drug, or entheogen, or medicinal mushroom dosage form; nano-dimensional drug delivery system structures of a psychedelic drug, entheogen, or medicinal mushroom delivering effective, stable dosage forms by intraoral, or sublingual, or buccal, or gastrointestinal, or transdermal, or intranasal, or rectal, or vaginal routes of administration; nano-dimensional drug delivery structures combining a self-emulsifying drug delivery system (SEDDs) with a nano-milled psychedelic drug, or entheogen, or medicinal mushroom for delivery across intestinal mucosa barriers; fast-acting intranasal or Intraoral delivered nano-dimensional drug delivery structures of a psychedelic drug, entheogen, or medicinal mushroom; nano-dimensional drug delivery system structures for improving pharmacokinetic characteristics of a psychedelic drug, entheogen, or medicinal mushroom dosage form.

This disclosure teaches compositions and methods of natural product extraction, isolation, separation, dephosphorization, stabilization, and manufacturing a stable dosage form of psilocin or psilocybin.

This disclosure teaches compositions and methods of natural product extraction, isolation, separation, stabilization, and manufacturing a stable dosage form of a psychedelic drug, or entheogen, or medicinal mushroom extract.

This disclosure teaches compositions and methods manufacturing stable dosage forms of a psychedelic drug, or entheogen, or medicinal mushrooms extract in a nano-dimensional drug delivery structure as a powder, fluid, crystalline, gel, or another drug dosage form.

This disclosure teaches compositions and methods of manufacturing stable dosage forms of psilocin or psilocybin in a unified process of production.

This disclosure teaches compositions and methods of manufacturing stable dosage forms of psychedelic drugs, or entheogen, or medicinal mushroom extracts in a nano-dimensional drug delivery structure in a unified process of production.

This disclosure teaches compositions and methods of dephosphorylation of psilocybin to psilocin and stabilization of a psilocin drug dosage form for therapeutic applications.

This disclosure teaches compositions and methods of manufacturing standardized dosage forms of psilocin or psilocybin in a nano-dimensional drug delivery structure in a unified process.

This disclosure teaches compositions and methods of manufacturing standardized dose forms of psychedelic drugs, or entheogens, or medicinal mushroom extracts in a nano-dimensional drug delivery structure in a unified process.

This disclosure teaches compositions and methods of formulation of psilocin drug dosage forms in a nano-dimensional drug delivery structure comprised of molecules with increased bioavailability or greater therapeutic activity.

This disclosure teaches compositions and methods of psychedelic drugs or entheogens, or medicinal mushrooms dosage forms comprised of molecules, phytochemicals, or compounds in a nano-dimensional drug delivery structure with increased bioavailability or greater therapeutic activity.

This disclosure teaches compositions and methods of a nano-dimensional -drug delivery structure system for improving pharmacokinetic characteristics of psilocin or psilocybin, including area under the curve (AUC) of plasma concentration-time profile, maximum plasma concentration (Cmax), time to maximum plasma concentration (Tmax) and volume of distribution (Vd or Vss), elimination half-life (t½), and clearance (CL).

This disclosure teaches compositions and methods of formulating a nano-drug delivery system for improving pharmacokinetic characteristics of a psychedelic drug, entheogen, or medicinal mushroom extract, including area under the curve (AUC) of plasma concentration-time profile, maximum plasma concentration (Cmax), time to maximum plasma concentration (Tmax) and volume of distribution (Vd or Vss), elimination half-life (t½), and clearance (CL).

This disclosure teaches compositions and methods of manufacturing a fast-acting intranasal delivered stable psilocin dosage form in a nano-dimensional drug delivery structure as an alternative to ingestion of slower-acting psilocybin.

This disclosure teaches compositions and methods of manufacturing a fast-acting intraoral buccal and sublingual delivered stable psilocin in a nano-dimensional drug delivery structure as an alternative to ingestion slower-acing psilocybin

This disclosure teaches compositions and methods of manufacturing a fast-acting intranasal delivered psychedelic drug, entheogen, and medicinal mushroom dosage form in a nano-dimensional drug delivery structure.

This disclosure teaches compositions and methods of manufacturing a fast-acting intraoral sublingual and buccal delivered psychedelic drug, entheogen, and medicinal mushroom dosage form in a nano-dimensional drug delivery structure.

This disclosure teaches compositions and methods of formulating medicinal mushrooms, or psychedelic drugs, or entheogens into a nano-dimensional delivery system structure combining nano-milling and a self-emulsifying drug delivery system (SEDDS) for gastrointestinal administration and delivery across gut mucosa.

This disclosure teaches compositions and methods for delivering more effective doses of a psychedelic drug, or entheogen, or medicinal mushroom in a nano-dimensional delivery system structure for gastrointestinal administration and delivery across gut mucosa by combining nano-milling and a self-emulsifying drug delivery system (SEDDS).

This disclosure teaches compositions and methods of formulating and delivering a dose of psilocin in a nano-dimensional delivery system structure into the body with a fast onset of activity, and greater bioavailability by intranasal transmucosal delivery.

This disclosure teaches compositions and methods of formulating and delivering a dose of psilocin in a nano-dimensional delivery system structure into the body with a fast onset of activity, and greater bioavailability by transmucosal sublingual or buccal delivery.

This disclosure teaches compositions and methods of formulating and delivering a dose of a psychedelic drug, or entheogen, or medicinal mushroom extract into the body with greater bioavailability by oral routes of administration.

This disclosure teaches compositions and methods of formulation for delivering a dose of a psychedelic into the body with greater bioavailability by oral, or sublingual mucosa, or buccal mucosa, or transdermal, or nasal, or ocular, or rectal, or vaginal or injection routes of administration.

This disclosure teaches compositions and methods of formulating permeation enhancers of psilocin or psilocybin for increasing dose delivery and therapeutic activity.

This disclosure teaches compositions and methods of formulating permeation enhancers of a psychedelic drug, or entheogen, or medicinal mushroom extract in a nano-dimensional delivery system structure for increasing dose delivery and therapeutic activity.

This disclosure teaches compositions and methods of formulating bioavailability enhancers of psilocin or psilocybin for increasing bioavailability and bio-efficacy and enabling reduced dosages.

This disclosure teaches compositions and methods of formulating bioavailability enhancers of a psychedelic drug, or entheogen, or and medicinal mushroom extract in a nano-dimensional delivery system structure for increasing bioavailability and bio-efficacy and enabling reduced dosages.

### DETAILED DESCRIPTION OF THE DISCLOSURE

Unless otherwise indicated, all numbers expressing quantities of ingredients, dimensions, reaction conditions, and so forth used in the specification and claims are to be understood as being modified in all instances by the term “about”.

[In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

### Definitions of This Disclosure

The terms “nano-drug delivery system” and “nano-dimensional drug delivery structure” in the present disclosure refer to a drug delivery system or structure in which at least one dimension is in the range of nanometer and formulated to deliver a psychedelic drug, or entheogen, or medicinal mushroom. As well as delivering phytochemicals, drugs, nutraceuticals, chemicals molecules, elements and compounds into the body targeted sites. A list includes polymer-based, non-polymeric, and lipid-based drug delivery structures and systems that include nanospheres, dendrimers, solid lipid nanoparticles, nanostructured lipid compositions, micelles, reverse micelles, nanogels, protein nanoparticles, carbon nanotubes, metallic nanoparticles, quantum dots, silica-based nanoparticles, liposomes, ethosomes, nanospheres, hydrogels, organogels, lipid-polymer nanostructures, nanoemulsions, self-emulsifying drug delivery systems self-nanoemulsifying drug delivery systems, and self-microemulsifying drug delivery systems.

The term “nano-dimensional in the present disclosure refers to drug delivery structures in which at least one dimension is less than 1 mcg in size and preferable has a mean diameter range from 1 to 500 nm.

### Nano-Dimensional Drug Delivery System and Structure Active Ingredient Terms

The term “psychedelic drugs” in the present disclosure refers to molecules or compounds or phytochemicals or botanicals or fungi or mushrooms that induce states of altered consciousness, perception, or thought, often with a heightened awareness of sensory input. Psychedelic drug classes include tryptamines, phenethylamines, and lysergamides, entheogens containing different psychedelic molecules. The term psychedelic drugs of this disclosure include synthetic forms, or derivatives, or analogs of psychedelic drug compounds.

Examples of tryptamine psychedelic drugs include base tryptamines aET, aMT, DALT, DET, DiPT, DMT, DPT, EiPT, EPT, MALT, MET, MiPT, MPT, NMT, PiPT); and substituted tryptamines⇒Ibogaine, Noribogaine, Ibogamine, Psilocin (4-HO-DMT), Psilocybin (4-PO-DMT) (4-AcO-DALT, 4-AcO-DET (Ethacetin), 4-AcO-DiPT (Ipracetin), 4-AcO-DMT (Psilacetin), 4-AcO-DPT (Pracetin), 4-AcO-EPT (Epracetin), 4-AcO-MALT, 4-AcO-MET (Metacetin), 4-AcO-MiPT (Mipracetin), 4-AcO-MPT (Mepracetin), 4-HO-DALT, 4-HO-DET (Ethocin), 4-HO-DiPT (Iprocin), 4-HO-DPT (Procin), 4-HO-EPT, (Eprocin), 4-HO-McPT, 4-HO-MET (Metocin), 4-HO-MALT, 4-HO-MiPT (Miprocin), 4-HO-MPT (Meprocin), 4-HO-PiPT, 4-MeO-MiPT, 5-Br-DMT, 5-Cl-aMT, 5-Cl-DMT, 5-MeO-aMT, 5-MeO-aET, 5-MeO-DALT, 5-MeO-DET, 5-MeO-DiPT (Foxy), 5-MeO-DMT, 5-MeO-DPT, 5-MeO-EiPT, 5-MeO-MALT, 5-MeO-MET, 5-MeO-MiPT (Moxy), 5-MeO-PiPT, Baeocystin (4-PO-NMT), Bufotenin (5-HO-DMT), α-MT, α-ET, MET, NMT, DPT, DiPT, 4-OH-DPT), AMT, 4-AcO-DMT and 4-AcODiPT,

Examples of phenethylamine psychedelic drugs include MDMA, MDA, mescaline, derivatives (3C-P, Allylescaline, Escaline, Isoproscaline, Methallylescaline, Proscaline and TMA), βk-2C-B, βOH-2C-B (BOHB), 2C-B-AN, 2C-B-FLY, 2C-D-5-EtO, 4C-D (ARIADNE), BOD (β-MeO-2C-D), Bromo-DragonFLY, HOT-7, Jimscaline, TMA TMA-1, MA-2, TMA-6, ZDCM-04, 2C-x substituted dimethoxyphenethylamine family, N-Benzyl phenethylamines, DOx substituted family. Phenethylamine psychedelics include compounds described in Alexander Shulgin's books PIKHAL AND the Shulgin Index: Psychedelic Phenethylamines and Related Compounds.

Examples of psychedelic lysergamide drugs include LSD (lysergic acid diethylamide LSD-25). (LSM-775), ergoline LSA, ALD-52, 1P-LSD, 1B-LSD 1cP-LSD, ETH-LAD, 1P-ETH-LAD, PRO-LAD, AL-LAD, PARGY-LAD, iso-LSD, MiPLA, LSH, LSZ, LSM-775, 1B-LSD, 1cP-AL-LAD, 1cP-LSD, 1cP-MiPLA, 1P-ETH-LAD, 1P-LSD, 1P-MiPLA, 1V-LSD, ALD-52 (1A-LSD), AL-LAD, Cabergoline, EiPLA, ETH-LAD, LAE-32, LSA (LA-111), LSH, LSM-775, LSZ, MiPLA, PARGY-LAD and PRO-LAD.

The term “entheogen” in the present disclosure refers to psychoactive substances often derived from plants that induce alterations in perception, mood, consciousness, cognition, or behavior The term entheogen was coined as a replacement for the terms hallucinogen and psychedelic. Examples of psychedelic entheogens include acacia confuse, amanita muscaria, ayahuasca, banisteriopsis caapi, cannabis, changa, datura, Hawaiian Baby Woodrose, Iboga, Mimosa hostilis, Morning glory, Psilocybin mushrooms, peyotyl, San Pedro, peyote, salvia divinorum, Syrian rue and yopo, and kava pyrones, and the entheogenic compounds described in the book Pharmacotheon by Jonathan Out/. The term entheogen in the present disclosure also includes derivatives, or analogs, or synthetic forms of entheogenic compounds.

The term “psilocybin mushrooms” in the present disclosure refers to fungi containing psilocybin or psilocin. It refers to the flowering body and mycelium of more than 180 mushroom species containing psilocybin and psilocin. That encompasses biological genera containing psilocybin mushrooms of Copelandia, Gymnopilus, Inocybe, Panaeolus, Pholiotina, Pluteus, and Psilocybe.

The term “prodrug” in the present disclosure refers to psychedelic drug compounds, entheogens, phytochemicals, nutraceuticals and drug substances that are inactive in the intended pharmacological actions and must be converted into the pharmacologically active agent by metabolic or physico-chemical transformation. Prodrugs encompass phytochemicals, botanical constituents endogenous substances, and end compounds resulting from synthetic and semisynthetic processes. Examples of prodrugs include psilocybin, psiloacetin and norpsilocin that are all produrgs of psilocin. Ibogaine, bufotenine, 2C-B-AN, and THC-A are; prodrugs of noribogaine, DMT, and THC. 1P-LSD, 1B-LSD, and ALD-52 are all prodrugs of LSD.

The term “medicinal mushrooms” in the present disclosure refers to species of mushrooms or fungi, mostly in the form of extracts or powder for prevention of diseases, or treatment of diseases, or nutritional application reasons. It refers to mushroom species that include Hericium erinaceus (Lion's Mane), Ganoderma lucidum (Reishi, Lingzhi), Trametes (Coriolus) versicolor (Turkey Tail), Grifola frondosa (Maitake, Hen of the Woods), Lentinula edodes (Shiitake), Agaricus blazei (Royal Sun Agaricus), Cordyceps sinensis (Caterpillar Fungus), Fomitopsis pinicola (Red Belted Polypore), Coprinus comatus (Shaggy Mane), Monascus purpureus (Red Yeast Rice), Polyporus umbellatus (Umbrella Polypore), Piptoporus betulinus (Birch Polypore) and Pleurotus ostreatus (Oyster Mushroom). The term medicinal mushrooms in the present disclosure also include derivatives, or analogs, or synthetic forms of medicinal mushroom compounds

### “Nano-Dimensional Drug Delivery Structure & Formulation Component Terms

The term “lipids” in the present disclosure refers to fatty, waxy, or oily compounds that are soluble in organic solvents and insoluble in polar solvents such as water. They are components, ingredients, and structural materials in the formulations, nano-dimensional drug delivery systems, and nano-dimensional structure of this disclosure. Lipids encompass fats and oils (triglycerides or triacylglycerols), fatty acids phospholipids, waxes, and steroids.

Fats and oil lipids are esters of glycerol and three fatty acids. Examples include lauric acid, myristic acid, palmitic acid, stearic acid, arachidic acid, palmitoleic acid, oleic acid, linoleic acid, linolenic acid arachidonic acid, fatty acid alcohols such as stearyl alcohol, cetyl alcohol, myristyl alcohol, lauryl alcohol; triglycerides such as trimyristin, tripalmitin, trilaurin; di; and triglycerides mixtures such as Suppocire NC, witepsol bases, glyceryl monostearate, glyceryl behenate, palmitostearate; and others such as cacao butter, castor oil, anhydrous milk fat, and hydrogenated palm oil.

Wax lipids include beeswax, cetyl palmitate, carnauba wax, cannabis wax extract;

Phospholipid lipids are a triester of glycerol with two fatty acids and one phosphate ion. They are components, ingredients, and structural materials in the formulations, nano-dimensional drug delivery systems and nano-dimensional structure of this disclosure Phospholipids include natural-occurring phospholipids such lecithins, phosphatidylcholine sphingosine, gangliosides, and phytosphingosine and combinations thereof derived from soy and lecithin that are preferable for use in this disclosure; synthetic phospholipids such as diacylglycerols, phosphatidic acids, phosphocholines, phosphoethanolamines, and phosphoglycerols; and essential phospholipids that refers to the highly purified extract of a specific fatty acid composition of phospholipids distinguished by their particularly high content of polyunsaturated fatty acids, predominantly linoleic acid (approximately 70%), linolenic acid, and oleic acid and with more than 75% of (3-sn-phosphatidyl) choline.

Besides phosphatidylcholine molecules, the essential phospholipid fraction includes phosphatidylethanolamine, phosphatidylinositol, and other lipids. Essential phospholipids include both hydrogenated and non-hydrogenated phospholipids manufactured from soy and sunflowers available from Lipoid and other suppliers such as Lipoid Phospholipons (90 G, 90 H, 85 G, H 90), Lipoid (S 75, S 40, S 80, E 80), and Lipoid Phosal (75 SA and 53 MCT).

The term “polymer” in the present disclosure refers to natural or synthetic substances composed of very large molecules, called macromolecules, that are multiples of simpler chemical units called monomers, They are components, ingredients, and structural materials in the formulations, nano-dimensional drug delivery systems and nano-dimensional structure of this disclosure. Examples of polymers include natural polymers such as arginine, chitosan, dextrin, polysaccharides, poly (glycolic acid), poly (lactic acid), and hyaluronic acid. Synthetic polymers such as poly (2-hydroxyethyl methacrylate), poly(N-isopropyl acrylamide)s, poly(ethylenimine). Bio-absorbable polymers like hydrogels such as poly (lactic acid) and poly (glycolic acid), and their copolymers. Bio-absorbable polymers like hydrogels such as poly (lactic acid) and poly (glycolic acid), and their copolymers. Poly lactic-co-glycolic acid, polyglycolic acid, Poly(N-isopropylacrylamide), dextran, Poly(N-isopropylacrylamide) polyethylenimine derivatives, polyethylenimine copolymers, and polyethylenimine conjugated bio-reducible polymers.

The term “chemical stabilizer” in the present disclosure refers to a chemical, formulation, compound, and molecule to prevent degradation, and/or create stability of psychedelic drugs, or entheogens, or medicinal mushrooms, and their constituents phytochemicals, chemicals, molecules, and compounds. They are components and ingredients in the formulations, nano-dimensional drug delivery systems, and nano-dimensional structure of this disclosure. Examples of classes of the chemical stabilizer include antioxidants, chelators, spin traps, acidifiers, ionic pairs, preservatives, and physical barriers coatings.

The term “mucoadhesive agent” in the present disclosure refers to agents molecules, chemicals, compounds that prolong the residence time of the nano-dimensional delivery system structures of this invention containing a psychedelic drug, or entheogen, or medicinal mushroom, at the sites of application that include the buccal and oral mucosa, skin and epidermal barriers, nasal mucosa, intestinal mucosa, vaginal mucosa, and rectum. They are components and ingredients in the formulations, nano-dimensional drug delivery systems, and nano-dimensional structure of this disclosure. Examples of mucoadhesive agents include gums, mucin starches, chitosan, pectins, polymers, polaxamers, cellulose derivatives (methylcellulose, ethylcellulose, hydroxyethylcellulose, hydroxyl propyl cellulose, hydroxyl propyl methylcellulose, sodium carboxymethylcellulose, poly (acrylic acid) polymers (carbomers, polycarbophil), poly (hydroxyethyl methyl acrylate), poly (ethylene oxide), poly (vinyl pyrrolidone), poly (vinyl alcohol), (ethylene oxide), p (vinyl pyrrolidone), and poly (vinyl alcohol).

The term “permeation enhancer” (also called absorption enhancers, sorption promoters, or accelerants) in the present disclosure refers to chemicals, compounds, molecules, or techniques used to improve the transport of a psychedelic drug or medicinal mushroom compound across the intestinal epithelium, through the skin, across the blood-brain barrier, across sublingual mucosa, across buccal mucosa, across nasal mucosa, or across a cell membrane. They are components and ingredients in the formulations, nano-dimensional drug delivery systems, and nano-dimensional structure of this disclosure. Examples of chemical permeation enhancers include including sulphoxides (such as dimethylsulphoxide, DMSO), Azones (e.g. laurocapram), pyrrolidones (for example 2-pyrrolidone, 2P), alcohols and alkanols (ethanol, or decanol), glycols (for example propylene glycol), fatty acids, terpenes, cyclodextrins, amines, and amides). It also physical methods used to deliver or transport micro and macromolecules of drugs, and other compounds across the skin, or other cell membranes that may increase their bioavailability, or bio-activity. Physical methods include iontophoresis, electroporation, sonophoresis, laser radiation and radiofrequency, microneedles and ablation.

The term “surfactant” in this disclosure refers to compounds that lower the surface tension (or interfacial tension) between two liquids or between a liquid and a solid, which act as emulsifiers, dispersants, wetting agents, and viscosity modifiers. They are components and ingredients in the formulations, nano-dimensional drug delivery systems, and nano-dimensional structure of this disclosure. In one embodiment, surfactants refer to amphiphilic molecules that are manufactured by chemical processes or are purified from natural sources or processes that can be anionic, cationic, zwitterionic. and nonionic.

Examples of anionic surfactants include fatty acid salts (“soaps”), sulfates (sodium dodecyl sulfate, ammonium lauryl sulfate, and other alkyl sulfate salts, sodium laureth sulfate), ether sulfates (alkyl ether sulfates, phosphate esters and sulphonates (alkyl benzene sulphonates).

Examples of cationic surfactants include amine salt, alkyl amine salt, alkyl diamine salt, ammonium salt, alkyl trimethyl ammonium salt, and benzalkonium chloride (bac)

Examples of zwitterionic surfactants include quarternary amine group and a carboxyl group containing surfactant (alkyl betaine, alkyl imidazoline), quarternary amine group and a sulfonic group containing surfactant (alkyl sulphobetaine), phospholipids surfactant (phosphatidyl serine, phosphatidyl choline. phosphatidyl ethanolamine), and carbohydrate-based surfactant (alkyl polyglucoside, alkyl glucamide).

Examples of nonionic surfactants include alkyl ethoxylate, nonylphenol ethoxylate, amine ethoxylate, alkyl poly(ethylene oxide alkylphenol poly(ethylene oxide), copolymers of poly(ethylene oxide), and poly(propylene oxide) (commercially called poloxamers or poloxamines), fatty alcohols, cetyl alcohol, oleyl alcohol, cocamide MEA, cocamide DEA, polysorbates: Tween 20, Tween 80, and dodecyl dimethylamine oxide.

The term “bioavailability enhancer” in the present disclosure refers to compounds and molecules used in combination with a psychedelic drug or medicinal mushroom extract molecule to increase their bioavailability and bioefficacy enabling reduced dosages. They are components and ingredients in the formulations, nano-dimensional drug delivery systems, and nano-dimensional structure of this disclosure. They function in ways that include: increasing delivery across the membrane, increasing bioavailability, potentiating their molecular conformational interaction, acting as receptors for their molecule, and making target cells more receptive to drugs.

The term “organic acids” in the present disclosure refers to organic compounds that possess acidic properties. They are components and ingredients in the formulations, nano-dimensional drug delivery systems, and nano-dimensional structure of this disclosure. Examples of organic acids include ascorbic oxalic acid, citric acid, tartaric acid, succinic acid, fumaric acid, and malic acid, glycolic acid, oxalic acid, acetic acid, acrylic acid, pyruvic acid, malonic acid, propanoic acid, hydroxypropanoic acid, lactic acid. glyceric acid. fumaric acid. maleic acid, oxaloacetic acid, crotonoic acid, acetoacetic acid, 2-oxobutanoic acid, methylmalonic acid, succinic acid, malic acid, L-tartaric acid, DL-tartaric acid, meso-tartaric acid, dihydroxytartaric acid, butanoic acid. isobutanoic acid. hydroxybutanoic acid, itaconic acid, mesaconic acid, oxoglutaric acid, glutaric acid, methylsuccinic acid, valeric acid, isovaleric acid, pivalic acid, phenol, cis-aconitic acid, trans-aconitic acid, ascorbic acid, citric acid, isocitric acid, adipic acid, caproic acid, benzoic acid, salicylic acid, gentisic acid, protocatechuic acid, gallic acid, cyclohexanecarboxylic, pimelic acid, phthalic acid, isophthalic acid, terephthalic acid, phenylacetic acid, toluic acid, m-toluic acid, p-toluic acid, mandelic acid. homogentistic acid, suberic acid, octanoic acid, cinnamic acid, nonanoic acid.

The term “natural product drugs” in the present disclosure refers to bioactive components or phytochemicals or drug compounds or drug precursors, or organic compounds isolated from natural sources that are produced by the pathways of primary or secondary metabolism. That encompasses organic compounds synthesized by a living organism or anything produced by living organisms that may be extracted from the cells, tissues, and secretions of microorganisms, fungi, plants, and animals.

The term “natural psychedelic medicine” in the present disclosure refers to psychedelic drug molecules or compounds derived from or contained in plants, fungi, mushrooms, microorganisms, sea vegetation, algae, animals. and trees.

### Delivery System and Extraction Terms

The term “self-emulsifying drug delivery systems (SEDDS)” in this disclosure refers to lipid-based formulations that encompass isotropic mixtures of natural or synthetic oils, solid or liquid surfactants, and co-surfactants. When exposed to aqueous media of gastrointestinal fluids undergo self-emulsification to form oil-in-water nanoemulsions or microemulsions. SEDDS are usually referred to as self-nanoemulsifying drug delivery systems (SNEDDS) or self-microemulsifying drug delivery systems (SMEDDS) depending on the nature of the resulting dispersions formed following their dilution. They are components in the formulations and nano-dimensional drug delivery systems of this disclosure.

The terms “milling and nanomilling” in this disclosure refer to methods in the formulation of nano-dimensional psychedelic drugs, entheogens, medical mushrooms, phytochemicals, and compounds by which an external force is applied to a solid that leads to its break-up into smaller particles. In one embodiment milling refers to wet grinding carried out using methods as a roller ointment mill, tumbling ball mill, vibratory ball mill, a planetary ball mill, a centrifugal fluid mill, an agitating beads mill, a flow conduit beads mill, an annular gap beads mill, and wet jet mill. In one embodiment. milling refers to dry grinding by compression or by friction, using methods as a jet mill, a hammer mill, a shearing mill, a roller mill, a shock shearing mill, a ball mill, and a tumbling mill. In one embodiment milling refers to wet processes for preventing the condensation of the nanoparticles so formed, and obtaining highly dispersed nanoparticles

The term “dephosphorylation” in the present disclosure refers to the removal of a phosphate (PO43-) group from the prodrug psilocybin to form psilocin by enzymatic and chemical agents.

The term “natural product extraction” in the present disclosure refers to natural product drug extraction techniques for extraction, or isolation of phytochemical or drug compounds from a natural product from using separation by adsorption or column chromatography, or partition chromatography, or counter-current chromatography, or gel filtration chromatography, or ion-exchange chromatography, or preparative gas chromatography, solvents, sonication, or supercritical fluid chromatography, simulated moving bed chromatography, or multi-dimensional chromatographic, or molecular distillation, or molecular imprinted technology, or membrane filtration characterized as microfiltration, ultrafiltration, and nanofiltration.

The terms “ultrasonication and sonication” in this disclosure refer to methods in the assembly of nanostructured carriers or nanoparticles from ultrasound waves generated from a sonicator in which ultrasonic amplitudes generate intense cavitation by alternating high-pressure and low-pressure cycles. These disperse and break up particles down to the nanometer scale. In one embodiment, ultrasonication refers to high-power ultrasonic liquid processors also known as sonicators, ultrasonic homogenizers, sonochemical reactors, ultrasonic mixers, and ultrasonic wet-milling systems.

The term “ultrasonic-assisted extraction” in this disclosure refers to an extraction process assisted with the usage of ultrasound waves generated from a sonicator passing through a liquid solvent containing solid particles to extract bioactive compounds.

The term “homogenization” in this disclosure refers to methods of formulation of drugs, or chemicals of high-shear fluid processing reducing the size of droplets and particles in liquid-liquid dispersions to submicron sizes. In one embodiment, homogenization refers to high shear fluid processors such as the Ultra-Turrax, Kinematika Polytron, Ross, and Silverson processors used for particle reduction, and liquid mixing. In one embodiment, homogenization refers to high-pressure homogenization piston gap and microfluidization methods such as APV Gaulin, Avestin, and Microfluidics homogenizers used for making nano-structures and nanoparticles.

The term “top-down” in the present disclosure refers to technology or methods of breaking down larger structures to generate sub-micron or nano-sized dimensional structures or nano-drug delivery systems from them. It also refers to slicing or successive cutting of material to get nano-sized particles, nanostructures, and nanocomposites. Examples of top-down technology include mechanical milling, three roller milling, jet milling, and sonication.

The term “bottom-up” in the present disclosure refers to technology or methods of forming single atoms and molecules into nano-sized particles or nanostructures or nanocomposites or nano-drug delivery systems. Examples of bottom-up nanofabrication technology include sedimentation and reduction techniques, sol-gel, green synthesis, spinning, and biochemical synthesis, It also refers to fabrication technologies forming lipid nanoparticles, polymer nanoparticles, lipid and polymer nanostructure and matrixes, hydrogels, nanogels, organogels, micelles, reverse micelles, premade and self-forming nanoemulsions, microemulsions, ethosomes, nanospheres, nano-dimensional lipid compositions, and nano-dimension structures or matrixes

### Delivery Routes and Administration Terms

The terms “transdermal delivery” and transdermal delivery systems” in the present disclosure refer to delivery systems designed to deliver a psychedelic drug, or entheogen, or medicinal mushroom, and other active ingredients across the skin and into systemic circulation. Ingredients penetrate through the stratum corneum and pass through the deeper epidermis and dermis. When they reach the dermal layer, they pass into the bloodstream at a controlled rate via dermal microcirculation.

The terms “buccal delivery” and “buccal delivery systems” in the present disclosure refer to delivery systems designed to deliver a psychedelic drug, or entheogen, or medical mushroom and other active ingredients by intraoral delivery across the buccal mucosa (the epithelium lining of the cheeks, gums, and lips) into the bloodstream. Effective buccal delivery systems require a mucoadhesive agent to increase mucosal retention time and absorption.

The terms “sublingual delivery” and “sublingual delivery systems” in the present disclosure refer to delivery systems designed to deliver a psychedelic drug, or entheogen, or medical mushroom and other active ingredients by intraoral delivery across under the tongue into the bloodstream. Effective sublingual delivery systems require a mucoadhesive agent to increase mucosal retention time and absorption before salivary washout carries the active ingredients down your throat.

The terms “intranasal delivery” and “intranasal delivery systems” in the present disclosure refer to delivery systems designed to deliver a psychedelic drug, or entheogen, or medical mushroom and other active ingredients across nasal mucosa into the bloodstream. Intranasal delivery includes direct nose-to-brain delivery systems along the olfactory and trigeminal that bypass the restrictive blood-brain barrier preventing most substances from entering the brain. Effective intranasal delivery systems require a mucoadhesive agent to increase mucosal retention time and absorption, and prevent ciliary motion carrying active ingredients down your throat,

The terms “gastrointestinal delivery” and gastrointestinal delivery systems” in the present disclosure refer to delivery systems designed to deliver a psychedelic drug, or entheogen, or medical mushroom and other orally administered active ingredients down your throat to the intestinal mucosa for absorption into the bloodstream, The oral route is by far the most common route of drug administration.

The term “route of administration” in the present disclosure refers to the path or delivery route or location by which or psychedelic drug, entheogen, or medical mushroom, or nutraceutical, or drug, or dietary supplement, or phytochemical, or molecule, or compound or other substance is taken into the body. Routes of administration refer to oral, gastrointestinal, intraoral (sublingual, buccal), transdermal, topical, intranasal, inhalation, nebulization, vaporization, ocular, vaginal, rectal, injection (intravenous, intramuscular, subcutaneous), transmucosal, among other routes of administration.

### Pharmacology and Pharmacokinetic Terms

The term “dosage form” in the present disclosure refers to the physical form of a dose of a or psychedelic drug, entheogen, or medicinal mushroom, including any chemical compound used as a drug, or medication intended for administration or consumption. Dosage forms comprise pills, tablets, capsules, oral suspensions, tinctures, emulsions, liquids drinks, powders, gels, creams, lotions, liniments, sprays, suppositories, crystals, aerosols, vaporization, liquid injections, transdermal liquids or gels or patches, eye drops, eye gels, eye ointments, nasal sprays, nasal gels, nasal solutions, oral strips, mucoadhesive buccal or sublingual compositions, among many others in the present disclosure. The route of administration delivery is dependent on the dosage form of the substance.

The term “effective dose” in the present disclosure refers to a dose or concentration of a psychedelic drug, or entheogen, or medicinal mushroom, or nutraceutical, or petrochemical or natural compound, or bioactive substance that produces a biological response.

The term “drug stability” in the present disclosure refers to the ability of drug dosage form to maintain physical, chemical, therapeutic, and microbial properties during the time of storage and patient use.

The term “degradation” in the present disclosure refers to the incapacity or incapability of a particular psychedelic drug, medicinal mushroom, or any formulation to remain within a particular chemical, microbiological, therapeutical, physical & toxicological specification.

The term “liquid dosage form” in the present disclosure refers to a solution, suspension, multiphase dispersion, syrup, gel, emulsion, nanoemulsion, liquid nanostructure, reconstituted powder, liquid preparation, liquid composition, liquid drug formulation, and liquid drug preparation.

The term “ bioactive substance” in the present disclosure refers to a substance or compound or molecules having an effect on, or causing a reaction, or triggers a response in living tissue or on a living organism, presenting therapeutic potential.

The term “physiochemical ” in the present disclosure refers to both physical and chemical properties, changes, and reactions. The ability of a chemical compound to elicit a pharmacological/therapeutic effect is related to the influence of various physical and chemical (physicochemical) properties of the chemical substance on the bio-molecule with which it interacts.

The term “dispersions” in the present disclosure refers to multi-phase mixtures consisting of substances that are insoluble in one another. One phase is the continuous phase in which small particles of the other phase are dispersed.

### Methods and Compositions of Stabilized Psychedelic Drugs and Medicinal Mushrooms Dose Forms

Effective drug dosage forms are required to maintain their physical, chemical, therapeutic, microbiological, and toxicological stability within their stated shelf-life.

Psilocin, psilocybin, naturally derived drugs and medicinal mushrooms are especially susceptible to chemical, physical, and microbiological degradation. Psilocin is highly unstable and degrades quickly in the presence of air, heat, and/or light and in solution.

Degradation is more problematic for natural product drugs made from natural product drug extraction techniques. During the extraction process, their bioactive compounds can degrade over time in different aqueous and ethanol solutions, as fluid solutions, dispersions, tinctures, emulsion, and powdered extractions. They can degrade over time in current delivery dose forms after manufacturing, during storage, and before use.

This disclosure teaches compositions and methods preventing three pathways of drug degradation to formulate a stable drug dosage form of psilocin, or psychedelic drugs, entheogens, or medicinal mushrooms.

The chemical degradation pathways include hydrolysis/solvolysis, oxidation, photolysis, polymerization, dehydration, isomerization, racemization, and chemical incompatibilities.

The physical degradation pathways include polymorphism, particle size, vaporization, evaporation, temperature, efflorescence, hygroscopy, and deliquescence.

The microbial degradation pathway of natural psychedelic drugs and medicinal mushrooms due to micro-organisms can render them harmful to the patient, or have an adverse effect on their properties.

This discourse teaches methods and compositions that maintain a stable drug dosage form of psychedelic drugs, or entheogens, or medicinal mushroom species. That is critical because drug degradation often leads to loss of potency and therapeutic effects.

This disclosure teaches compositions and methods of psychedelic drugs, or entheogens, or medicinal mushrooms as stabile drug dosage forms for therapy.

This disclosure teaches compositions and methods for preventing physiochemical degradation of psilocin with a stabilizer chemical or protective molecular structure.

This disclosure teaches compositions and methods for preventing physiochemical degradation of a psychedelic drug, or entheogen, or medicinal mushroom with a stabilizer or protective molecular structure.

In one embodiment of this disclosure, the protective molecular structure is a nanoparticle.

In one embodiment of this disclosure, the protective molecular structure is a lipid or polymer structure.

In one embodiment of this disclosure, the protective molecular structure is a lipid or phospholipid.

In one embodiment of this disclosure, the protective molecular structure is a polymer.

In one embodiment of this disclosure, the protective molecular structure is an emulsion, or nanoemulsion, or microemulsion, or self-forming nanoemulsion, or self-forming microemulsion.

In one embodiment of this disclosure, the protective molecular structure is a film system.

In one embodiment of this disclosure, the protective molecular structure is a microparticle.

In one embodiment of this disclosure, the protective molecular structure is a dendrimer system.

In one embodiment of this disclosure, the stabilizer is a chemical added to the growth medium of mushroom mycelium of psilocybin-containing mushrooms or medicinal mushrooms.

In one embodiment of this disclosure, the stabilizer is a polymer, or macromolecule, or shellac applied to particles or tablets of a psilocybin of psilocin dosage form.

In one embodiment of this disclosure, the stabilizer is a polymer or macromolecule or shellac applied to particles or tablets of a psychedelic drug, or entheogen, or medicinal mushroom extract dosage form.

In one embodiment of this disclosure, the stabilizer is an organic acid.

In one embodiment of this disclosure, the stabilizer is an antioxidant.

In one embodiment of this disclosure, the stabilizer is a phytochemical.

In one embodiment of this disclosure, the stabilizer is a lipid.

In one embodiment of this disclosure, the stabilizer is a phospholipid.

In one embodiment of this disclosure, the stabilizer is a polymer.

In one embodiment of this disclosure, the stabilizer is a spin trap compound.

In one embodiment of this disclosure, the stabilizer is a chelation agent.

In one embodiment of this disclosure, the stabilizer is a preservative compound.

In one embodiment of this disclosure, the stabilizer is a chemical constituent of Psilocybe cubensis.

In one embodiment of this disclosure, the stabilizer is nanoparticle structure.

In one embodiment of this disclosure, the stabilizer is hydrogel.

In one embodiment of this disclosure, the stabilizer is an organogel.

In one embodiment of this disclosure, the stabilizer is a micelle.

In one embodiment of this disclosure, the stabilizer is a reverse micelle.

In one embodiment of this disclosure, the stabilizer is nanoparticle lipid composition.

In one embodiment of this disclosure, the stabilizer is a nanoemulsion.

In In one embodiment of this disclosure, the stabilizer is an emulsion.

### Compositions and Methods for Dephosphorylation of Psilocybin to Psilocin and Stabilized Psilocin Dosage Forms

Both psilocybin and psilocin are phytochemical constituents of Psilocybe cubensis, also known as “magic mushrooms”. Psilocin is the therapeutic active form of the prodrug psilocybin. It has more than 200 times the affinity for serotonin 5-HT2A receptors than psilocybin. Psilocin is lipophilic and crosses the restrictive blood-brain barrier. Psilocybin is water-soluble and has a hard time passing the blood-brain barrier.

Ingesting psilocybin is merely the means for administering a psilocin equivalent. Psilocybin and psilocin are stoichiometrically equivalent in potency. One molecule of psilocybin undergoes dephosphorylation in the intestinal tract to form a molecule of psilocin, which provides the majority of the therapeutic psychoactive effects.

Drug dose forms of psilocybin, psilocin, and psychedelic mushroom extracts will be required to maintain physical, chemical, therapeutic, microbiological, and toxicological stability within their stated shelf-life when they are available. However, psilocybin, psilocin, psychedelic mushrooms, medicinal mushrooms, entheogens, and naturally-derived psychedelic drugs are susceptible to degradation.

A full spectrum fluid extraction form of Psilocybe cubenis and other psilocybin-containing mushroom species can contain alkaline phosphatase enzymes as phytochemical constituents that dephosphorylate the psilocybin molecules into unstable psilocin molecules that may degrade before therapeutic use.

Furthermore, the administration route for psilocybin dose forms necessitates oral administration to be therapeutically effective. Enabling stomach acid and intestinal alkaline phosphatase enzyme's dephosphorylation of psilocybin to form psilocin, the biological active therapeutic molecular form.

Administration of a psilocybin dosage form by non-GI routes of administration will have a minimum degree of efficacy. Only a minuscule amount of bioactive psilocin may be formed from the dephosphorylation of psilocybin by the kidneys and blood.

This disclosure teaches composition and methods of dephosphorylation of psilocybin to a stabile dose form of psilocin. A formulation process that is scalable and commercially applicable to manufacturing psilocin dosage forms for therapeutic applications.

This disclosure teaches compositions and methods for formulating nano-dimensional drug delivery system structures of psilocin dosage forms.

This disclosure teaches compositions and methods for administering a psilocin dosage form in a nano-dimensional delivery system structure into the body by nasal mucosal, or nasal direct to brain, or transdermal, or sublingual mucosa, or buccal mucosa, or oral, or injectable routes of administration.

This disclosure teaches compositions and methods of dephosphorylation of psilocybin to psilocin by a chemical compound.

This disclosure teaches compositions and methods of dephosphorylation of psilocybin to psilocin by an organic acid or compound.

This disclosure compositions and method for dephosphorylation of psilocybin to psilocin and stabilization of a psilocin drug dose form in a unified process of production.

This disclosure teaches compositions and methods of aqueous-organic, or organic, or solvent, or liquid-liquid, or acid-base, or percolation, or soxhlet or reflux or super-critical fluid or pressurized liquid, or microwave-assisted, of sonication-assisted, or pulsed electric field, or enzyme-assisted or steam distillation or hydro-distillation extraction technology for formulating a stabilized drug dosage form of psilocin for therapy.

This disclosure teaches compositions and methods of aqueous-organic, or organic, or solvent, or liquid-liquid, or acid-base, or percolation, or soxhlet or reflux or super-critical fluid or pressurized liquid, or microwave-assisted, or sonication-assisted, or pulsed electric field, or enzyme-assisted, or steam distillation or hydro-distillation extraction technology for formulating a stabilizing a dosage form of a psychedelic drug, or entheogen, or medicinal mushroom.

This disclosure teaches compositions and methods of a natural product drug extraction technique of psilocin combining the use of heat and organic acid for the dephosphorylation of psilocybin to a psilocin dosage form in a unified process of production.

This disclosure teaches compositions and methods combining an organic acid and sonication-assisted extraction of Psilocybe cubensis mushrooms for extraction and dephosphorization of psilocybin to psilocin.

This disclosure teaches compositions and methods of combining extraction, dephosphorylation, and stabilization of psilocin in a unified process to produce a stable, effective drug dosage form for therapeutic application.

This disclosure teaches compositions and methods for dephosphorylation of psilocybin to psilocin by acids into a stable psilocin drug dosage form for therapy.

### Methods and Compositions of Delivery Systems for Psychedelic Drugs, Entheogens, and Medicinal Mushrooms

Many psychedelic drugs, entheogens, and medicinal mushroom extracts have poor pharmacokinetic characteristics that can limit their clinical effectiveness. Including a low area under the curve (AUC) of plasma concentration-time profile, maximum plasma concentration (Cmax), time to maximum plasma concentration (Tmax), and volume of distribution (Vd or Vss). As well as a poor elimination half-life (t½), and clearance (CL).

Many psychedelic drugs, entheogens, and medicinal mushroom extract compounds despite their impressive in-vitro findings demonstrate negligible in-vivo activity. This is due to their poor solubility or improper molecular size resulting in poor absorption and hence poor bioavailability as well difficulty in passing the BBB barrier.

All medicinal mushrooms contain immune-supporting β-glucan polysaccharides embedded in their cell walls made from an indigestible unabsorbable fibrous material called “chitin”. Consequentially β-glucan has less than 5% bioavailability.

Various components of a psychedelic drug, or entheogen, or medical mushroom may contribute to synergistic effects. Isolation, purification, and separation of bioactive ingredients can lead to a partial loss of specific activity due to the removal of chemically related substances contributing to the activity of the main components. Often the chemical complexity of the extract is important for the bioavailability and bioactivity of their active components.

This disclosure teaches compositions and methods of formulating a nano-dimensional drug delivery system structured for improving pharmacokinetic characteristics of psilocin or psilocybin, including area under the curve (AUC) of plasma concentration-time profile, maximum plasma concentration (Cmax), time to maximum plasma concentration (Tmax) and volume of distribution (Vd or Vss), elimination half-life (t½), and clearance (CL).

This disclosure teaches compositions and methods of formulating a nano-dimensional drug delivery system structure for improving pharmacokinetic characteristics of a psychedelic drug, or entheogen, or medicinal mushroom extract, including area under the curve (AUC) of plasma concentration-time profile, maximum plasma concentration (Cmax), time to maximum plasma concentration (Tmax) and volume of distribution (Vd or Vss), elimination half-life (t½), and clearance (CL).

This disclosure teaches compositions and methods of formulating permeation enhancers in nano-dimensional delivery system structures of psilocin or psilocybin for increasing dose delivery and therapeutic activity.

This disclosure teaches compositions and methods of formulating permeation enhancers in nano-dimensional delivery system structures of a psychedelic drug, or entheogen, or medicinal mushroom for increasing their dose delivery and therapeutic activity.

This disclosure teaches compositions and methods of formulating bioavailability enhancers of psilocin or psilocybin for increasing their bioavailability and bio-efficacy and reducing the dose required for therapy.

This disclosure teaches compositions and methods of formulating bioavailability enhancers of a psychedelic drug, or entheogen, or medicinal mushroom extract for increasing bioavailability and bioefficacy, and reducing the dose required for therapy.

In one embodiment of this invention, the bioavailability enhancer is a phospholipid-based drug delivery system.

In one embodiment of this invention, the bioavailability enhancer is a nano-dimensional drug delivery system.

In one embodiment of this invention, the bioavailability enhancer is a permeation enhancer.

This disclosure teaches compositions and methods combining a natural product drug extraction technique and nano-dimensional-drug delivery system manufacturing in a unified process for producing a psilocin or psilocybin drug dose form.

This disclosure teaches compositions and methods combining a natural product drug extraction technique and nano-dimensional drug delivery system manufacturing in a unified process for producing a psychedelic drug, or entheogen, or medicinal mushroom extract drug dose form.

One embodiment of this invention teaches methods and compositions of a nano-dimensional-drug delivery system structure for manufacturing a psychedelic drug, or entheogen, or medicinal mushroom dose form with increased bioavailability for gastrointestinal delivery.

One embodiment of this invention teaches methods and compositions combining a nano-dimensional drug delivery system and permeation enhancer for manufacturing a psilocin drug dose form with increased bioavailability.

One embodiment of this invention teaches compositions and methods combining a nano-dimensional drug delivery system and permeation enhancer for manufacturing a psychedelic drug, or entheogen, or medicinal mushroom extract dose form with increased bioavailability.

One embodiment of this invention teaches compositions and methods for manufacturing a nano-dimensional drug delivery system of a psilocin drug dose form with increased bioavailability for sublingual, or buccal delivery into the body.

One embodiment of this invention teaches a nano-dimensional drug delivery system for manufacturing a psilocin dose form for nasal delivery into the bloodstream.

One embodiment of this invention teaches a nano-dimensional drug delivery system for manufacturing a psilocin dose form for nasal delivery directly into the brain along the olfactory nerve or trigeminal nerve.

One embodiment of this invention disclosure teaches compositions and methods for manufacturing a nano-dimensional drug delivery system for a psychedelic drug, or entheogen or medicinal mushroom dose form with increased bioavailability for sublingual, or buccal delivery to the bloodstream.

One embodiment of this invention disclosure teaches compositions and methods for manufacturing a nano-dimensional drug delivery system for a psychedelic drug, or entheogen, or medicinal mushroom extract dose form for nasal delivery directly into the brain along the olfactory nerve or trigeminal nerve.

One embodiment of this invention disclosure teaches compositions and methods of a nano-dimensional drug delivery system for manufacturing a psychedelic drug, or entheogen, or medicinal mushroom extract dose form with increased bioavailability for intranasal delivery to the bloodstream.

One embodiment of this invention disclosure teaches compositions and methods of a nano-dimensional drug delivery system for manufacturing a psychedelic drug, or entheogen, or medicinal mushroom extract dose form with increased bioavailability for gastrointestinal delivery to the bloodstream,

One embodiment of this invention teaches compositions and methods of a nano-dimensional drug delivery system for manufacturing a psychedelic drug, or entheogen, or medicinal mushroom extract dose form with increased bioavailability for transmucosal vaginal or rectal delivery to the bloodstream.

This disclosure teaches compositions and methods of manufacturing a nano-dimensional drug delivery system structure for a psychedelic drug, or entheogen, or medicinal mushroom extract by combining one or more top-down and one or more bottom-up processes of fabrication or assembly.

The disclosure teaches compositions and methods of manufacturing a nano-dimensional drug delivery system for a psilocin or psilocybin dose form for increasing their absorption or bioavailability by combining one or more top-down and one or more bottom-up processes of fabrication of assembly.

The disclosure teaches compositions and methods of manufacturing a nano-dimensional drug delivery system of a psychedelic drug dosage form for increasing their absorption or bioavailability by combining one or more top-down and one or more bottom-up processes of fabrication of assembly.

This disclosure teaches compositions and methods of combining nano-milling and self-emulsifying drug delivery of a psychedelic drug, or entheogen, or medicinal mushroom extract dosage form for improving intestinal absorption or bioavailability.

### EXAMPLE 1 Psilocin Extraction, Dephosphorization, and Stabilization

This example teaches composition and method for a natural product extraction process of psilocin (4-hydroxy-NN-dimethyltryptamine) and psilocybin (4-phosphoryloxy-NN-dimethyltryptamine) from Psilocybe cubensis, dephosphorization of the prodrug silocybin to psilocin, and stabilizing a psilocin dosage form for therapy. Psilocin is the active drug form of the pro-drug psilocybin.

1. 200 gms of dried Psiloybe cubensis mushrooms is ground in a grinder to a fine powder (40 mesh).
2. The mushroom powder is added into a 5000 ml jacketed beaker attached by tubing to a chiller and peristaltic for cooling.
3. 2000 liters of distilled water is discharged into the jacketed beaker mushroom powder beaker followed by 2 gms of 50:50 ratio of acetic and ascorbic acids.
4. Beaker stood for 1 hour.
5 The aqueous mushroom and organic acid blend is sonicated for 25 minutes by a 40 mm probe with 2500 watts of power at 20 khz with cooling at 50° C. to extract psilocybin and psilocin from the mushrooms and dephosphorization of psilocybin to psilocin. An overhead paddle stirrer at 300 RPM is used to transport the mushroom particles uniformly to the probe.
6. After sonication, mushroom solids are removed by filtration and pressing from extracted phytochemicals. Water, ethanol and organic acids are removed by rotor-evaporation.
7. HPLC analysis for psilocin and psilocybin is conducted. The extract weights for psilocin and psilocybin based on 1 gm of crude Psilocybe cubensis mushrooms is determined.

| | |
|---|---|
| Psilocin | 1.41 mg/gram |
| Psliocybin | .32 mg/gram |

### EXAMPLE 2 Erinacine Extraction from H. erinaceus mycelium powder (Lions Mane)

This example teaches composition and method for an efficient natural product extraction process of bioactive Erinacines from H. erinaceus mycelium powder (Lions Mane).

1. 500 gms of dried H. erinaceus mycelium is ground in a grinder to a fine powder (40 mesh).
2. The mushroom powder is added to 5000 ml jacketed beaker attached by tubing to a chiller and peristaltic for cooling.
3. 2000 liters of a 70:30 ratio of ethanol and distilled water is discharged into the beaker of mushrooms.
4 The aqueous mushroom blend is sonicated for 45 minutes by a 40 mm probe with 2500 watt of power at 20 khz and cooling at 50° C. to extract Erinacines. An overhead paddle stirrer at 300 RPM is used to transport the mushroom particles uniformly to the probe.
5. After completion of the extraction, the sample is vacuum filtered and then centrifuged for 5 minutes. A rotary evaporator is used for water elimination from the supernatants.
6. HPLC analysis of Erinacines is conducted. The extract weight for Erinacines based on 1 gm of crude Lion's Mane is determined.

| | |
|---|---|
| Erinacine | 2.9 mg/gram |

### EXAMPLE 3 Intranasal Delivered NanoPsilocin

This example teaches composition and method for a manufacturing process for incorporating the sonicated-assisted psilocin extract of Example 1 into a nano-dimensional lipid-polymer mucoadhesive drug delivery structure in a unified process of production as a stable dosage form for intranasal delivery.

1. Phase I is prepared to contain ethanol, water, psilocin extract, propylene glycol, Pluronic F127, Polysorbate 20, dimethyl isosorbide, pectin, d-limonene, ascorbates, and I-menthol.
2. Phase II phase is prepared with lecithin (phospholipids), medium-chained triglycerides, and oleic acid.
3. Ingredient amounts were adjusted to formulate a nanostructured psilocin dosage form containing 2.5 mg of psilocin per 100 MCL.
4. 200 ml of Phase I and II blend are discharged into a jacketed beaker attached by tubing to a chiller and peristaltic for chilling.
5. The 2 phases are mixed by a high shear homogenizer for 15 minutes at 8,000 RPM under cooling.
6. The phases is sonicated for 12 minutes by a 40 mm probe with 2500 watt of power at 20 khz with cooling at approx. 50° C. An overhead paddle stirrer at 300 RPM was used to transport fluid uniformly to the probe.

7. The finished product of nano-structured psilocin full-spectrum extract is packaged in proprietary 5 ml airless pump nasal dispensers discharging 25 mg of psilocin per 100 MCL per pump.

8. Four test subjects ages 25 to 60 are administered 2 pumps intranasally with their head tilted back 45 degrees. Subjects were instructed to pinch nostrils together after dispensing.
9. Onset times of psychotropic effects in each subject that correlate with psilocin bioavailability and delivery to the brain are recorded. Subjects report a duration of psychotropic effects lasting 2.5 to 3.5 hours and peak effects occurring at 50-70 minutes.

| Subject | Onset Time |
|---|---|
| 1 | 9 minutes |
| 2 | 14 minutes |
| 3 | 7 minutes |
| 4 | 10.5 minutes |

The average onset time of intranasal delivered NanoPsilocin is 10.125 minutes. By contrast, the reported onset time of effects from ingesting psilocybin is reported to typically begin after 30 minutes.

### EXAMPLE 4 Intraoral Delivered Psilocin

This example teaches composition and method for a manufacturing process for incorporating the sonicated-assisted psilocin extract of Example 1 into a nano-dimensional lipid-polymer mucoadhesive drug delivery structure in a unified process of production as a stable dosage form for intraoral delivery.

1. Phase I is prepared to contain ethanol, water, psilocin extract, propylene glycol, Pluronic F127, Polysorbate 20, dimethyl isosorbide, pectin, d-limonene, ascorbates, and I-menthol,
2. Phase II phase is prepared with lecithin (phospholipids), medium-chained triglycerides, and oleic acid.
3. Ingredient amounts are adjusted to formulate a nanostructured psilocin dosage form delivering 12.5 mg of psilocin per 110 MCL.
4. 200 ml of Phase I and II blend are discharged into a jacketed beaker attached by tubing to a chiller and peristaltic for chilling.
5. The 2 phases are mixed by a high shear homogenizer for 15 minutes at 8,000 RPM under cooling.
6. The phases are sonicated for 12 minutes by a 40 mm probe with 2500 watts of power at 20 khz with cooling at approx. 50° C. An overhead paddle stirrer at 300 RPM was used to transport fluid uniformly to the probe.
7. The finished product of nano-structured psilocin full-spectrum extract is packaged in proprietary 5 ml airless lotion-type pump dispensers discharging 12.5 mg of psilocin per 110 MCL.
8 Four test subjects ages 24 to 49 are administered 2 pumps between their lower cheek and gums by the applicator and used fingertip motion to spread over a larger area for transmucosal absorption.
9. Onset times of psychotropic effects in each subject that correlate with psilocin bioavailability and delivery to the brain are recorded. Subjects report a duration of effect lasing 2.5 to 3.5 hours with peak effects occurring around 60 minutes.

| Subject | Onset Time |
|---|---|
| 1 | 9 minutes |
| 2 | 14 minutes |
| 3 | 7 minutes |
| 4 | 10.5 minutes |

The average onset time of intraoral buccal delivered NanoPsilocin is 11 minutes. By contrast, the reported onset time of effects from ingesting psilocybin is reported to typically begin after 30 minutes.

### EXAMPLE 5 Transdermal Delivered Psilocin

This example teaches composition and method for a manufacturing process for incorporating the sonicated-assisted psilocin extract of Example 1 into a nano-dimensional lipidpolymer mucoadhesive drug delivery structure in a unified process of production as a stable dosage form for transdermal delivery.

1. Phase I is prepared to contain ethanol, water, psilocin extract, propylene glycol, Pluronic F127, Polysorbate 20, dimethyl isosorbide, pectin, d-limonene, ascorbates, and I-menthol,
2. Phase II phase is prepared with lecithin (phospholipids), medium-chained triglycerides, and oleic acid.
3. Ingredient amounts are adjusted to formulate a nanostructured psilocin dosage form delivering 12.5 mg of psilocin per 125 MCL.
4. 200 ml of Phase I and II blend are discharged into a jacketed beaker attached by tubing to a chiller and peristaltic for chilling.
5. The 2 phases are mixed by a high shear homogenizer for 15 minutes at 8,000 RPM under cooling.
6. The phases are sonicated for 12 minutes by a 40 mm probe with 2500 watts of power at 20 khz with cooling at approx. 50° C. An overhead paddle stirrer at 300 RPM is used to transport fluid uniformly to the probe.
7. The finished product of nano-structured Psilocin full-spectrum extract of Psilocybe cubensis is packaged in proprietary 5 ml airless pump pen dispensers discharging 12.5 mg of psilocin per 125 MCL per pump.
8. In Vitro skin permeation of transdermal nano-structured psilocin is tested by a one-chambered Franz Diffusion cell using Strat M pads. Comparing the flux (the amount of permeant crossing the membrane per time) between nano-structured psilocin full spectrum extract and psilocin full spectrum extract quantified by HPLC,
9. HPLC results find a 788% increased transport across the membrane from nano-structured psilocin full spectrum extract compared to a standard psilocin and psilocybin extract.

### EXAMPLE 6 Intranasal Delivered 5-MeO-DMT

This example teaches a composition and method of a manufacturing process for incorporating the tryptamine psychedelic drug 5-MeO-DMT (5-methoxy-N,N-dimethyltryptamine) and other psychedelic drugs into a nano-dimensional lipid-polymer mucoadhesive drug delivery structure in a unified process of production as a stable dosage form for intranasal delivery.

1. Phase I is prepared to contain ethanol, water, psilocin extract, propylene glycol, Pluronic F127, Polysorbate 20, dimethyl isosorbide, pectin, d-limonene, and I-menthol,
2. Phase II phase is prepared with lecithin (phospholipids), medium chained triglycerides, and oleic acid
3. Ingredient amounts are adjusted to formulate a nanostructured psilocin dosage form containing 20 mg of 5-MeO-DMT per 100 MCL. 200 ml of Phase I and II blend are discharged into a jacketed beaker attached by tubing to a chiller and peristaltic for chilling.
4. The 2 phases are mixed by a high shear homogenizer for 15 minutes at 8,000 RPM under cooling.
5. The phases are sonicated for 12 minutes by a 40 mm probe with 2500 watts of power at 20 khz with cooling at 50° C. An overhead paddle stirrer at 300 RPM is used to transport fluid uniformly to the probe.
6. The finished product of nano-structured 5-MeO DMT is packaged in proprietary 5 ml airless pump nasal dispensers discharging 20 mg of 5-MeO-DMT per 100 MCL per pump.
7. Four test subjects ages 20 to 45 administered 1 pump intranasally of 5-MeO-DMT with their head tilted back 45 degrees. Subjects are instructed to pinch nostrils together after dispensing.
8. Onset times of psychotropic effects in each subject that correlate with 5-MeO-DMT bioavailability and delivery to the brain were recorded. Subjects report a duration of psychotropic effects lasting 60 to 75 minutes and peak effects occurring at 35-45 minutes.

| Subject | Onset Time |
|---|---|
| 1 | 4 minutes |
| 2 | 7 minutes |
| 3 | 5.5 minutes |
| 4 | 4.5 minutes |

The average onset time of intranasal delivered 5-MeO-DMT is 5.25 minutes

### EXAMPLE 7 Intraoral Delivered Mescaline

This example teaches composition and method for a manufacturing process for incorporating the phenethylamine psychedelic drug Mescaline (5-trimethoxyphenethylamine), and other psychedelic drugs into a nano-dimensional lipid-polymer mucoadhesive drug delivery structure in a unified process of production as a stable dosage form for intranasal delivery.

1. Phase I is prepared to contain Mescaline HCL, water, propylene glycol, Pluronic F127, Polysorbate 20, Dimethyl Isosorbide, pectin, d-limonene, and I-menthol.
2. Phase II phase is prepared with lecithin (phospholipids), medium-chained triglycerides, and oleic acid.
3. Ingredient amounts are adjusted to formulate a nanostructured psilocin dosage form containing 25 mg of Mescaline per 110 MCL. 200 ml of Phase 1 and II blends are discharged into a jacketed beaker attached by tubing to a chiller and peristaltic for chilling.
4. The 2 phases are mixed by a high shear homogenizer for 15 minutes at 8,000 RPM under cooling.
5. The phases are sonicated for 12 minutes by a 40 mm probe with 2500 watts of power at 20 khz with cooling at approximately 50° C. An overhead paddle stirrer at 300 RPM is used to transport fluid uniformly to the probe.
6. The finished product of nano-structured Mescaline is packaged in proprietary 5 ml airless pump lotion-type dispensers discharging 25 mg of Mescaline per 110 MCL per pump.
7. Four test subjects ages 26 to 58 years of age take 8 pumps by buccal administration over 4 minutes, subjects dispense two pumps every minute for four times between their lower cheek and gums by the applicator. And use fingertip motion to spread over their entire gum area.
8. Onset times of psychotropic effects in each subject that correlate with Mescaline bioavailability, delivery to the brain, and biotransformation is recorded. Subjects report a duration of psychotropic effects lasting 8 to 10 hours and peak effects occurring around 120 to 150 minutes

| Subject | Onset Time |
|---|---|
| 1 | 23 minutes |
| 2 | 20 minutes |
| 3 | 19.5 minutes |
| 4 | 26 minutes |

The average onset time of intraoral delivered Mescaline is 22.15 minutes. By contrast, the reported onset time of effects from ingesting Mescaline is reported as 60-180 minutes.

### EXAMPLE 8 Gastrointestinal Delivered Red Reishi Mushroom Extract

This example teaches composition and method for a manufacturing process for nanomilling Red Reishi Mushroom (Ganoderma lucidum) Extract, and other medicinal mushrooms, psychedelic drugs, and entheogens, and incorporating them into a nano-dimensional self-emulsifying drug delivery system (SEDDS). delivery system structure for gastrointestinal delivery across the intestinal mucosa,

1. 1 kg of Red Reishi Mushrooms 30% polysaccharides extract powder is nanomilled in a 12″ jet mill to an average particle size distribution of less than 165 nm confirmed by laser light diffraction using a Malvern Zetasizer Nano.
2. A SEDDS is formulated by mixing ethyl linoleate and MCT as the oil phase, Cremophor EL as emulsifier, and ethyl alcohol as the co-emulsifier with water.
3. A visual assessment method is used to confirm the self-emulsification of nano-milled Red Reishi Mushroom extract in the SEEDS formulation in a 1:100 dilution in water over time.
4. The average droplet size and the size distribution of nano-milled Red Reishi Mushroom incorporated into a SEDDS are measured by a Malvern Zetasizer Nano and found to be 75 nm.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

The description of the present invention has been presented for purposes of illustration and description but is not intended to be exhaustive or limiting of the invention to the form disclosed. Many modifications and variations will be apparent to those of ordinary skill in the art. The embodiment described and shown in the was chosen and described in order to best explain the principles of the invention, the practical application, and to enable others of ordinary skill in the art to understand the invention for various embodiments with various modifications as are suited to the particular use contemplated.

While the compositions and methods that have been described in terms of what are presently considered to be the most practical and preferred embodiments, it is to be understood that the disclosure need not be limited to the disclosed embodiments. It is intended to cover various modifications and similar arrangements included within the spirit and scope of the disclosure, the scope of which should be accorded the broadest interpretation so as to encompass all such modifications and similar structures.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionaries recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

Support should be understood to exist to the degree required under new matter laws—including but not limited to United States Patent Law 35 USC 132 or other such laws—to permit the addition of any of the various dependencies or other elements presented under one independent or concept as dependencies or elements under any other independent concept.

To the extent that insubstantial substitutes are made, to the extent that the applicant did not in fact draft any disclosure, method, or composition so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” concept interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms as to afford the applicant the broadest coverage legally permissible.

The description of the present invention has been presented for purposes of illustration and description but is not intended to be exhaustive or limiting of the invention to the form disclosed. Many modifications and variations will be apparent to those of ordinary skill in the art. The embodiment described were chosen and described in order to best explain the principles of the invention, the practical application, and to enable others of ordinary skill in the art to understand the invention for various embodiments with various modifications as are suited to the particular use contemplated.

What is claimed is:

1. A nano-dimensional drug delivery compositional structure comprising:
psychedelic drug or entheogen or medicinal mushroom; lipids;
polymers; and
at least one ingredient selected from the group consisting of mucoadhesive, permeation enhancers, chemical stabilizers, and surfactants.

2. The nano-dimensional compositional structure of claim 1, wherein the structure is administered via the sublingual mucosa and buccal mucosa.

3. The nano-dimensional compositional structure of claim 1, wherein the structure is administered via the rectal or vaginal mucosa.

4. The nano-dimensional compositional structure of claim 1, wherein the structure is administered across nasal mucosal barriers.

5. The nano-dimensional compositional structure of claim 1, wherein the structure is administered from the nasal cavity into the brain bypassing the blood-brain barrier.

6. The nano-dimensional compositional structure of claim 1, wherein the structure is administered across nasal dermal and epidermal barriers.

7. A self-emulsifying drug delivery system comprising:
a nano-milled psychedelic drug or entheogen or medicinal mushroom extract; lipids; surfactants; and emulsifiers.

8. A natural extraction process for psilocybin mushrooms comprising:
combining, separating, dephosphorylating, and stabilizing psilocin molecules in a method of production.

9. The natural extraction process of claim 8, performed using sonication.

10. The natural extraction process of claim 8, performed using organic acids.

11. The nano-dimensional compositional structure of claim 1, wherein the psychedelic drug or entheogen or medicinal mushroom is present in the form of an extract, wherein the extract is prepared by an extraction process for separating, and stabilizing the psychedelic drug, or entheogen, or medicinal mushroom.

12. The nano-dimensional compositional structure of claim 11, wherein extraction process further comprises sonification.

13. The nano-dimensional compositional structure of claim 1, wherein the structure can deliver a stable, standardized, and precise amount of a psychedelic drug, or entheogen, or medicinal medicine for therapy.

14. The nano-dimensional compositional structure of claim 1, wherein the structure has a size distribution from 25 nm to 195 nm.

15. The nano-dimensional compositional structure of claim 1, wherein the nano-dimensional structure increases the bioavailability of psychedelic drug, or entheogen, or medicinal mushroom 2-fold to 10-fold compared to bioavailability without a nano-dimensional compositional structure.

16. The nano-dimensional compositional structure of claim 1, wherein the structure decreases than an amount of a psychedelic drug, or entheogen, or medicinal mushroom extract 2-fold to 8-fold needed to illicit the same therapeutic effect compared to the therapeutic effect without a nano-dimensional structure.

17. The nano-dimensional compositional structure of claim 1, wherein the structure decreases the onset time of effects from intranasal or intraoral delivered psilocin to less than 20 minutes after the time of administration.

18. The nano-dimensional compositional structure of claim 1, wherein the structure decreases the onset time of effect from an intranasal or intraoral delivered psychedelic drug, or entheogen, or medicinal mushroom extract to less than 20 minutes after the time of administration.
